(19) United States
(12) Patent Application Publication
(10) Pub. No.: US 2008/0031924 A1
(43) Pub. Date: Feb. 7, 2008

(54) Therapeutic device for delivering controlled air stream flow and/or photodynamic light therapy

(76) Inventors: Darcy J. Gilson, Morristown, NJ (US); James A. Gilson, Maywood, NJ (US); Julia A. DeBellis, Maywood, NJ (US)

Correspondence Address: WATOV & KIPNES P.C., P.O. Box 247, Princeton Junction, NJ, 08550, US

(21) Appl. No.: 11/497,635

(22) Filed: Aug. 2, 2006

(51) Int. Cl. A61F 13/00

(52) U.S. Cl. 424/433; 607/86

(57) ABSTRACT

A therapeutic device for treating and preventing dermatological diseases and conditions, includes a housing comprising a head portion having an opening through which light energy exits the device and a handle; a light generating assembly adapted to generate and direct a preselected type of light energy to the skin; and moisture removing assembly operatively associated with the housing for removing moisture from the surface of the skin being treated.

### FIELD OF THE INVENTION

The present invention relates to a therapeutic device, and more particularly to a therapeutic device for treating and preventing dermatological disease and conditions including, for example, skin irritation, wounds and infections through delivery of controlled air stream flow and/or light therapy.

### BACKGROUND OF THE INVENTION

In the medical field, especially in the field of pediatrics, there is an elevated rate of infection associated with the female genitalia and male perineum and rectal areas that result from the presence of persistent moisture. Typically, the presence of such moisture in the poorly ventilated anatomical areas can result from a number of causes, including poor and incomplete drying after bathing and/or use of moist wipes to clean the aforementioned areas. In the pediatric population, this condition is also a persistent problem when diaper changes are done with an incomplete drying of these anatomical areas.

In both the pediatric and the adult population, skin infection and irritation can result from the growth of a number of organisms and physical conditions including, but not limited to: viral warts, acne, psoriasis, cutaneous t-cell lymphoma, dermatoses, actinic keratoses (Bowen's Disease), basil cell carcinoma, scleroderma, hyperplasia oedenia, langerhans cell depletion, topical dermatitis, eczema, herpes simplex virus and labialis, acne vulgaris, lichen planus, cutaneous graft vs. host disease, papiloma virus, warts, apoptosis, vitiligo pityriasis, lichenoids, scleroderma condylomata, acuminata pemphigus (Hailey-Hailey disease), lichen sclerosis, condylomata, tissue repair, pain control, diabetic ulcers, and decubitus ulcers in elderly and disabled.

A number of these conditions are either exacerbated or caused by persistent moisture in and around specified anatomic areas in the male and female. Medical research has determined that peri-urethral vestibular bands, found in the female genital area can become inflamed and predisposed to infections as a direct result of incomplete drying and persistent moisture. Research has suggested that peri-urethral bands appear to be a risk factor for the development of vulvovaginitis because their presence is associated with erythema which results from the pooling of moisture. The erythema and inflammation associated with these bands are often mistaken for signs of child abuse by an untrained medical professional and can result in concomitant psycho-social trauma, including the removal of the child from the home.

Medical evidence exists that the inflammation of these bands and the female genital area can be prevented by thoroughly drying the female genital area during diaper changes and as a component of a medically prescribed hygiene regimen for children and adolescents. In adults, the aforementioned disease processes and conditions can be either caused or complicated by incomplete drying of the male and female genital areas. Even adults with no range of motion limitations can find reaching and adequately drying these anatomical areas difficult.

The utilization of light therapy to treat cutaneous diseases is well established in the literature. Additionally, the use of UV radiation has been shown to inhibit bacterial, fungal, viral and other growth. The combination of cooling air in combination with laser treatment is well established to maintain the laser treated skin portion at a lower cooling temperature. However, the combination of warming air for drying and light therapy has not been studied. There are numerous conditions where moisture is the cause of or exacerbates disease progression including dermatitis, candidiasis, genital herpes, and the like.

Light therapy can be germicidal, fungicidal and may even exhibit virucidal properties, which can improve or eliminate disease progression in many of the aforementioned medical diseases and conditions. Recent developments in the field of cancer treatment and therapeutics provide for the application of a controlled light source to activate or augment certain photosensitive agents that are applied topically for specific skin conditions, and cancers such as 5-Aminolaevulinic acid (ALA),methylene blue, hydrogen peroxide, toluidine blue O (TBO), 8-methoxypsoralen and other psoralens, and many others.

Accordingly, there is a need for a therapeutic device for delivering a controlled air stream flow and/or light therapy that provides a simple and economical method of directing controlled air stream and/or light energy to difficult to reach anatomical areas. The device would be used for reducing or eliminating the presence of persistent moisture, which can alleviate the development or complication of any of the aforementioned medical conditions, as well as prevent, for example, common dermatitis. There is also a need for a therapeutic device that can emit a therapeutically useful light energy to treat or prevent dermatological disease or conditions. There is a further need for a therapeutic device that can be utilized as a specifically controlled light source in conjunction with designated photosensitive therapeutic agents. There is a further need for a therapeutic device that is portable, hand-holdable and compact for unobstrusive use under clothing or diapers and on various parts of the body, and that can be used to treat or prevent dermatological diseases and conditions in an simple, effective and economical manner.

### SUMMARY OF THE INVENTION

The present invention is related to a therapeutic device that can deliver a controlled air stream flow and/or emit a therapeutically effective light energy to the surface of a skin for treatment or prevention of dermatological diseases or conditions. The therapeutic device of the present invention is adapted especially for treating the skin especially areas that typically susceptible to the presence of persistent moisture including, for example, bottom portions of the feet, underarm areas, regions of the female genitalia and male perineum and rectal areas. The therapeutic device of the present invention is constructed in the form of a compact, portable, and hand-holdable apparatus effective for implementing dermatological drying via controlled air stream flow and/or light therapy via emission of therapeutic light.

In one form, the therapeutic device of the present invention includes a moisture removing assembly for removing moisture from the surface of the skin being treated through the delivery of a controlled air stream flow. The controlled air stream flow may be regulated by its temperature and/or flow rate favorable for providing a drying therapeutic effect on the skin.

In another form, the therapeutic device of the present invention may include a light generating assembly adapted to generate and direct a preselected type of light energy to the surface of the skin being treated. Optionally, the therapeutic device may further include a moisture removing assembly for removing moisture from the surface of the skin being treated that can be operated alone or in combination with the light generating assembly.

In one aspect of the present invention, there is provided a therapeutic device for treating and preventing dermatological diseases and conditions, the device comprising:

a housing comprising a head portion and a handle portion; and

a light generating assembly located at the head portion, the light generating assembly adapted to generate and direct a preselected type of light energy to the surface of the skin being treated.

In another aspect of the present invention, there is provided a therapeutic device for treating and preventing dermatological diseases and conditions, the device comprising:

a housing comprising a head portion and a handle portion; and

a moisture reducing assembly located at the head portion, the moisture reducing assembly adapted to generate and direct a controlled air stream flow to the surface of the skin being treated at a temperature and flow rate conducive to safely drying the skin surface.

In a further aspect of the present invention, there is provided a therapeutic device for treating and preventing dermatological diseases and conditions, said device comprising:

a) a housing comprising a head portion having an opening and a handle;

b) a light generating assembly adapted to generate and direct a preselected type of light energy to the skin through the opening through which light energy exits the device; and

c) moisture removing means for removing moisture from the surface of the skin being treated.

### BRIEF DESCRIPTION OF THE DRAWINGS

The following drawings in which like reference characters indicate like parts are illustrative of embodiments of the invention and are not to be construed as limiting the invention as encompassed by the claims forming part of the application.

FIG. 1 is a side cross sectional view of a therapeutic device for treating and preventing dermatological disease and conditions for one embodiment of the present invention;

FIG. 2 is a rear elevational view of the therapeutic device in accordance with the present invention;

FIG. 3 is a side cross sectional view of a therapeutic device for treating and preventing dermatological disease and conditions comprising a light generating assembly for another embodiment of the present invention; and

FIG. 4 is a side cross sectional view of a therapeutic device for treating and preventing dermatological disease and conditions comprising a gas directing assembly for another embodiment of the present invention.

### DETAILED DESCRIPTION OF THE INVENTION

The present invention is directed to a therapeutic device for preventing and treating medical skin diseases and conditions including, but not limited to vulvo-vaginitis and related afflictions of the female and male genitalia, perineum and rectal areas, and other areas of the body prone to the presence of moisture. The therapeutic device of the present invention is designed to specifically prevent and treat dermatological diseases and conditions such as, for example, skin infections, particularly those in infants and adolescents, which can result from improper and incomplete hygiene or other diseases. In the infant, this can be referred to as diaper dermatitis and is often directly related to improper and incomplete hygiene when changing diapers.

In one embodiment of the present invention, the therapeutic device of the present invention in the form of a convenient hand held portable device comprises a gas directing assembly that provides heated air at a controlled temperature and flow rate for safely drying skin at or near body temperature. The present invention further provides a convenient hand held portable device that optionally provides heated air for drying skin at a temperature of from about 85 to 115° F. with a flow rate of from about 0.5 to 2.0 m/sec.

In another embodiment of the present invention, the therapeutic device of the present invention in the form of a convenient hand held portable device comprises a light generating assembly that provides light energy at therapeutically effective wavelengths for exposure of the patient's skin. The present embodiment can provide a therapeutic dose of light at a specific therapeutic wavelength or full spectrum light, or any combination thereof. The therapeutic wavelength can be selected from infrared light, ultraviolet light, blue light, red light, yellow light, orange light, green light, violet light and combinations thereof. The therapeutic device can be utilized to provide light therapy alone, or in combination with the heated air.

The therapeutic device of the present invention adapted to provide light emission can be used in combination with a photoactivating therapeutic agent applied to the surface of the patient's skin being treated. Such photoactivating therapeutic agent can be topically applied for specific skin conditions, and may include, for example, 5-aminolaevulinic acid (ALA), methylene blue, hydrogen peroxide, toluidine blue O (TBO), psoralens such as 8-methoxypsoralen, and the like.

The therapeutic device of the present invention may be adapted to provide a dose of light from about 0.5 to 5.0 J/cm⁻², which may be narrow band UVB (TL01) at 311 nm, UVB at 280-320 nm, UVA at 340-400 nm, a combination of UVA and UVB, infra-red, near infrared, blue light at 405 nm, and/or full spectrum light, or any combination thereof and can be utilized to provide light therapy in conjunction with heated air, heated air alone or light therapy alone.

The therapeutic device of the present invention can utilize a normal household plug socket 115 V or contain two 9 V rechargeable batteries which has a wall mountable recharging unit that can utilize a normal household plug socket 115 V. The power is designed for all components, including the heating element and the light source to run efficiently and pose no risk of injury due to electric shock, even if the user were to be exposed directly to the electrical system. Other design features as discussed below make the risk of electric shock virtually impossible.

The therapeutic device of the present invention is contained in a plastic housing and features a tapered, telescoping handle suitable for use by children five years old through adult. The device features a pivoting head, which makes it possible to utilize in hard to reach areas.

### Heating

The therapeutic device of the present invention can utilize commercial or custom made elements or bulbs including, but not limited to infrared, band, tubular, rod, silicone, bolt, stud, cartridge, such as Caloritech Cat. No. C1025801, band, strip, finned, drum or any combination thereof. The element may contain or consist of any material or combination thereof including but not limited to ceramic, metal, silicone, glass or any combination thereof.

### Light Dosing

The therapeutic device may utilize commercially available bulbs, or tubes such as (SOS Light Bulbs GPX5#129134 including narrow band UVB (TL01) at 311 nm, UVB at 280-320 nm, UVA at 340-400 nm, a combination of UVA and UVB, infra-red, near infrared, blue light at 405 nm, and or full spectrum light but not limited to any combination thereof, depending upon the application. The bulb or tube may contain or consist of any material or combination thereof including but not limited to mercury vapor ceramic, metal, silicone, glass or any combination thereof.

### Air Flow

The therapeutic device of the present invention can utilize an AC impellor or fan, which are commercially available, such as Sofasco Model A8025P requiring an air intake grate. The impellor or fan will be electronically regulated to produce the desired airflow into the path of the heating element allowing heated air to exit through the opening.

### Safety Features

In one embodiment, the therapeutic device of the present invention includes a temperature sensor which may consist of, but is not limited to a NTC Thermistor, Silicone PTC, Platinum RTD, IC Voltage (° C. or ° F), IC Current or Digital which may be housed by means of an epoxy coating, disc, glass encapsulated DO-35, TO-92, thin film, silicon or any other material, light sensor which may consist of a fiber optic probe or photocell made of Silicon, Silicon GaP, or GaAsP, custom made or commercially available similar to Skye SKR 1850 depending upon the wavelength being generated from the light source or a light dependent resistor connected to a circuit that produces voltage when it reaches the upper limit of the light dose and or intensity and automatically shuts off the light source, and/or timer that regulates the heating element and light source respectively to control the intensity and duration of light (dose) and air temperature and flow duration to prevent burning. The therapeutic device provides a physical mechanical light barrier for UV therapy such that adults can utilize this in the pediatric population prohibiting the child from viewing the source directly.

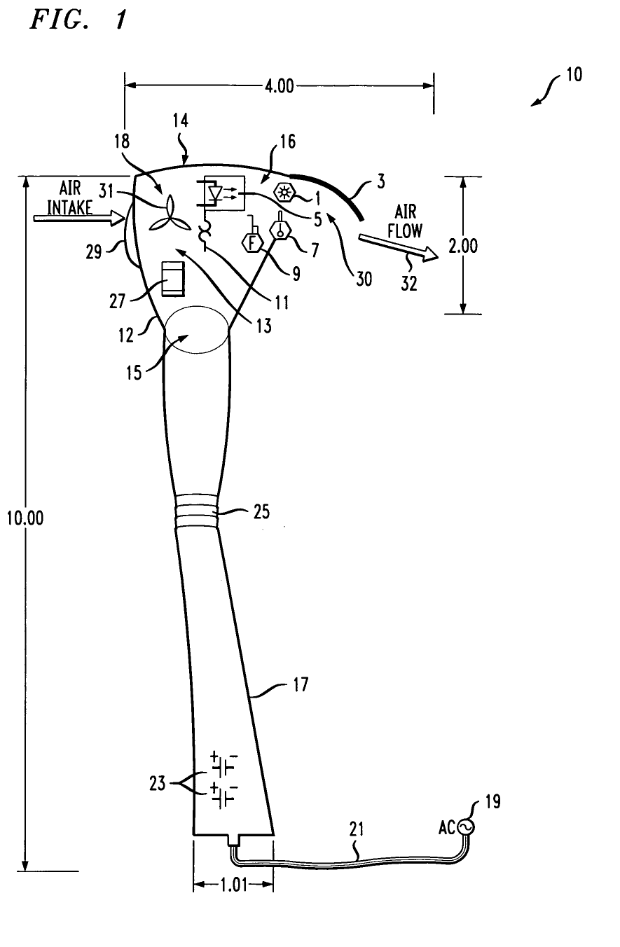

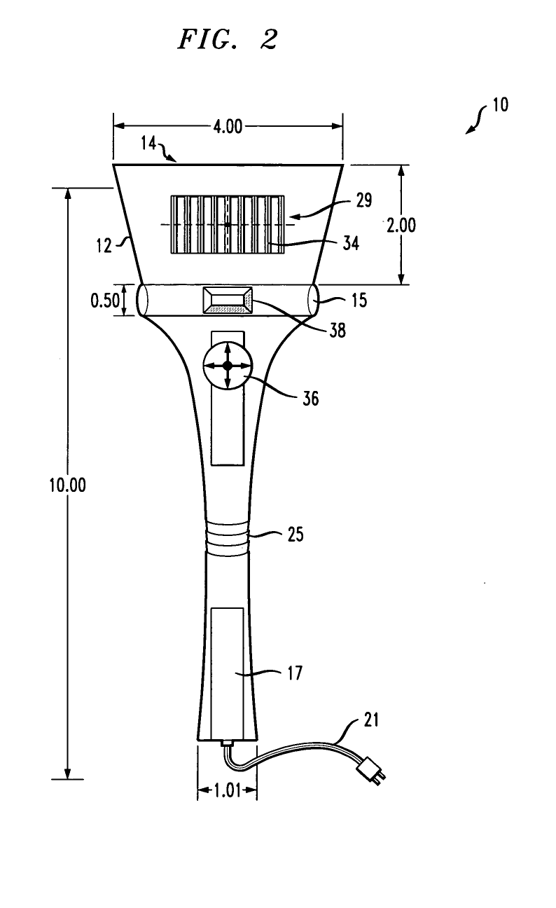

Referring to FIGS. 1 and 2, there is shown a therapeutic device identified by reference numeral 10 for one embodiment of the present invention. The device 10 includes a housing 12 comprising a head portion 14 and a handle 17. The handle 17 is configured to enable the user to hold the device 10 at a proper location during use.

The handle 17 may further include a telescoping mechanism 25 composed of a series of concentric rings (not shown) that can be extended by an activating button (not shown). The rings (not shown) include tabs that can lock each circle into place after the circle passes over them. The telescoping mechanism 25 can be retracted through the operation of the activating button. The telescoping mechanism 25 facilitates self administration by the patient where the device 10 is used at hard to reach places including the feet area, head area, genital area, anal area. The handle 17 may be adjustably configured for allowing adult and child users to comfortably hold the device 10 during use.

The head portion 14 of the housing 12 further comprises a light generating assembly 16, and optionally a gas directing assembly 18. The light generating assembly 16 includes a light transmitter 5 for emitting a preselected type of light energy which may be a narrowband or a single coherent light, and a light intensity or dose sensor 1 for measuring the intensity and dose of light being emitted from the device 10. The housing 12 further includes a light shield 3 to prevent exposure of the light transmitter 5 beyond the area of the skin to be treated. The light shield 3 further prevents direct exposure of the light to the eyes of the patient or those nearby.

The device 10 includes a circular pivot 15 located between the head portion 14 and the handle 17. The circular pivot 15 allows the head portion 14 to rotate up to about 270°, thus allowing the light generating assembly to be suitably oriented to project the light energy in hard to reach areas including the feet, back, head, genital and anal regions. The circular pivot 15 includes a depressible button 38 (see FIG. 2) that allows the head portion 14 to rotate to a desired angle which locks into place upon release of the button 38.

The device 10 further includes a circuit controller 27 comprising the necessary electrical components to regulate the light generating assembly 16 and the optional gas directing assembly 18. During operation, the dose sensor 1 ensures that the intensity of the light emitted from the light transmitter 5 does not exceed a preset upper limit. If the limit is passed, then the dose sensor 1 transmits a signal to the circuit controller 27 to reduce the power to the light transmitter 5, thereby regulating the intensity generated.

In the present embodiment, the head portion 14 of the housing 12 includes an air intake vent 29 with a protective grille 34 (see FIG. 2) to prevent entry of foreign objects, and an air exhaust vent 30 in connection with the gas directing assembly 18. The gas directing assembly 18 includes an air flow generator 31 for drawing air through the air intake vent 29 and direct it out through the air exhaust vent 30 in the form of a gas stream 32. The gas directing assembly 18 further includes a thermal element 11 for heating the air drawn in by the air flow generator to a preselected temperature, typically around the body temperature of the patient, a flow sensor 9 for measuring the flow rate of air generated by the air flow generator 31, and a heat sensor 7 for detecting the temperature of the gas stream 32.

During operation, the flow sensor 9 ensures that the flow rate remains within a preset limit, and exceeding such limits can cause the flow sensor 9 to transmit a flow signal to the circuit controller 27 to reduce or increase the power to the air flow generator 31, thereby modulating flow rate of the gas stream 32. Concurrently, the heat sensor 7 ensures that the temperature of the gas stream 32 remains within a preset limit, and exceeding such limits can cause the heat sensor 7 to transmit a heating signal to the circuit controller 27 to reduce or increase the power to the thermal element 11, thereby modulating the temperature of the gas stream 32.

The circuit controller 27 is electrically connected to the light transmitter 5, the air flow generator 31, the dose sensor 1, the flow sensor 9 and the heat sensor 7. The circuit controller 27 is programmed to provide constant power to the flow sensor 9, heat sensor 7 and dose sensor 1, and upon receiving feedback signals from the respective sensors 9, 7 and 1, appropriately adjust the voltage and power to the corresponding one of the air flow generator 31, the thermal element 11 and the light transmitter 5 to maintain the operation of the device 10 within preset parameters.

The interior of the housing 12 may be composed of a sufficient amount of a thermal insulator 13 to provide a heat barrier around the thermal element 11 and the light transmitter 5 to prevent the housing 12 from heating up to a temperature which can cause burns upon contact thereon.

The light generating assembly 16, the optional air directing assembly 16 and the circuit controller 27 are supplied with electrical power through an electrical cord 21 for connection to an AC power source 19 (e.g., electrical wall outlet). The device 10 may further include rechargeable battery sources 23 for enabling cord-less use.

As shown in FIG. 2, the device 10 includes a control dial 36 that can be rotated into different positions by the user for operation. By operating the control dial 36, the user can activate the light generating assembly 16 and the gas directing assembly 18 simultaneously, activating the light generating assembly 16 only, activating the light generating assembly 18 only, or deactivating the device 10.

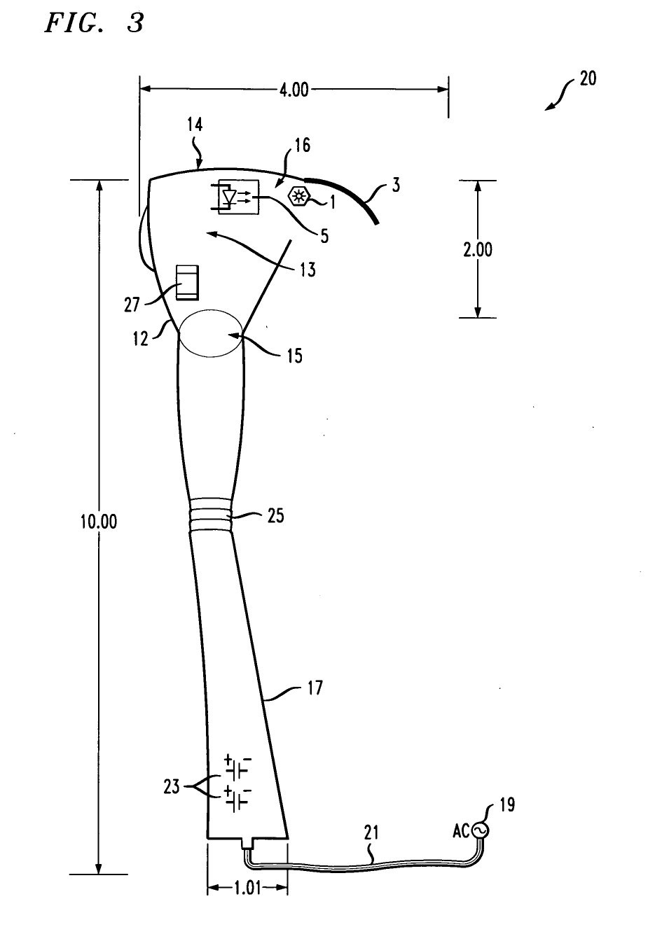

With reference to FIG. 3, there is shown a therapeutic device identified generally by reference numeral 20 for another embodiment of the present invention. The device 20 includes all the features of the device 10 except a gas directing assembly.

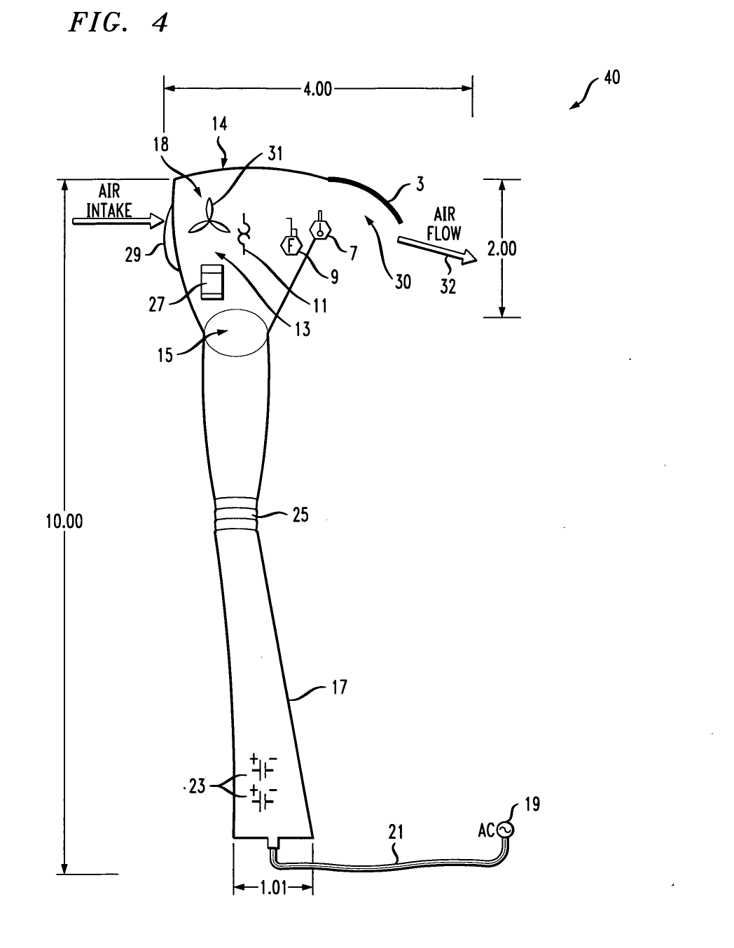

With reference to FIG. 4, there is shown a therapeutic device identified generally by reference numeral 40 for another embodiment of the present invention. The device 40 includes all the features of the device 10 except a light generating assembly.

With reference to FIGS. 1 and 2, the specific operation of the device 10 is specified.

Step 1. The user to grasp the handle 17 of the device 10 and remove the device 10 from a battery charger/holder (not shown).

Step 2. The user will then extend the handle 17 (if necessary) to the desired length in order to reach the anatomical area to be treated.

Step 3. The user will then depress the button 38 allowing the head portion 14 of the device 10 to rotate to the optimal angle for application to the affected area, and release the button 38.

Step 4. The user will then move the control dial 36 to the position which will turn the device 10 on and perform the selected respective function.

Step 5. The user will then using the handle 17 of the device 10 to maintain the head portion 14 of the device 10, not less than 1 inch from the surface of the anatomical area to be treated until the device 10 shuts of automatically. For extensive surface areas or multiple areas, the user will have to repeat Steps 2-5.

Step 6. After the treatment is completed the user moves the control dial 36 to the off position, which will turn the device 10 off.

Step 7. The user will depress the button 38 and rotate the head portion 14 of the device 10 to rotate back to the original angle and release the button 38.

Step 8. The user will retract the telescoping handle 17 by holding the top portion of the housing 12 with one hand and the bottom portion of the housing 12 with the other hand and squeezing together.

Step 9. The user will then return the device 10 to the re-charger/holder (not shown).

### EXAMPLE

### Making and Using the Photodynamic Light Therapy Device of the Present Invention

For one embodiment, the device of the present invention may be fabricated through the use of a four piece custom molded pieces for forming the housing with the appropriate openings for dials, cords, buttons, and the like. The molded pieces may also include an air intake grid or it can be purchased separately. Commercially available thermal insulation such as MICROTHERM® may be inserted and affixed in such a way as to cover all possible inside surfaces of the housing forming the head portion. A ratchet and pawl attached to the button similar to catalog no. SRT2/3-C may be used between the head and handle as pivot.

Two to four NiCad batteries such as described in the attachments will supply power to the electronics and fan motor. These batteries will be rechargeable and may fit into the handle of the device. The use of this device is initially planned to be subject to a medical protocol, developed by medical researchers, pediatricians and other medical personnel and entities who will write a carefully planned, effective and easy to use protocol for the device to be used by an adult when caring for a child or by anyone, age 5 and older who can benefit from the use of the device. In general, the device can be used for many different medical conditions or applications and can be manufactured to treat or prevent a specific medical condition, or multiple medical conditions. The device may therefore be referred to as “condition dependent”. The user will be instructed to pivot the head of the device in order to gain access to the anatomical area to be treated, turn on the heated airflow and hold the device a recommended distance from the skin around the area to be treated.

The user may be directed to pass the head of the device over and around the anatomical areas for the prescribed period of time and the device can be manufactured with a built in timer that automatically shuts off the heated airflow. The condition dependent use of a light source will be automatically limited by total dosage measured by the light sensor and or a pre-set timer. The only action that the user will need to be cognizant of will be directing the flow toward the proper anatomical area, which will be explained in detail with the use of medical condition dependent diagrams and a short teaching video that will come with the product.

If the device is held too close to the skin for too long a time, a temperature sensor/servo regulator will cycle the heating element off and an alarm light will activate. The user will be directed to retry the device after a set amount of time on which the skin can cool. A successful application cycle will be condition and type and size of the anatomical area dependent.

The instructions will call for the therapeutic use of this device anytime after the genital anatomy becomes wet. In children, this will be with every diaper change and in adults, this will be as necessary. For other medical condition specific or therapeutic uses detailed instructions and protocols for use will be provided with the device. In addition, the specific parameters for use of the device may be prescribed by a physician.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. A therapeutic device for treating and preventing dermatological diseases and conditions, said device comprising:
a housing comprising a head portion and a handle portion; and
a light generating assembly located at said head portion, said light generating assembly adapted to generate and direct a preselected type of light energy to the surface of the skin being treated.

2. The device of claim 1 further comprising moisture removing means for removing moisture from the surface of the skin being treated.

3. The device of claim 1 wherein moisture removing means further comprises:
an opening in the head portion through which a gas exits the device;
a warming assembly comprising means for generating a flow of the gas, and a heater for elevating the temperature of the gas to a predetermined level; and
a gas directing assembly including a conduit for directing the warmed gas toward the skin.

4. The device of claim 3 wherein the gas directing assembly comprises a pivot means enabling the head portion of the housing to pivot about an axis.

5. The device of claim 3 further comprising means for controlling the rate of flow of the warmed gas.

6. The device of claim 3 wherein the gas is an oxygen containing gas.

7. The device of claim 6 wherein the oxygen containing gas is air.

8. The device of claim 3 wherein the gas warming assembly heats the gas to a temperature of from about 85° F. to 115° F.

9. The device of claim 8 wherein the gas warming assembly heats the gas to a temperature of about 98.6° F.

10. The device of claim 3 wherein the gas warming assembly provides a flow rate of the warm gas of from about 0.5 to 2.0 m/sec.

11. The device of claim 3 further comprising a heat sensor and means for maintaining the warm gas within a desired temperature range.

12. The device of claim 1 wherein the light generating assembly comprises a light source and a light directing device for directing the generated light from the housing to the skin.

13. The device of claim 12 wherein the generated light is selected from the group consisting of light having a narrow band wavelength of 311 nm, 280-320 nm, 340 nm-400 nm, 405 nm-440 nm, infrared, near infrared and combinations thereof.

14. The device of claim 1 wherein the light source provides a dose of light in the range of from about 0.5 to 5.0 J/cm².

15. The device of claim 1 wherein the handle is a telescoping handle.

16. The device of claim 1 wherein the device is portable.

17. The device of claim 1 wherein the housing is configured to treat the areas of the skin associated with female the genitalia and adjacent areas, male perineum and anal area.

18. A therapeutic device for treating and preventing dermatological diseases and conditions, said device comprising:
a) a housing comprising a head portion having an opening and a handle;
b) a light generating assembly adapted to generate and direct a preselected type of light energy to the skin through the opening through which light energy exits the device; and
c) moisture removing means for removing moisture from the surface of the skin being treated.

19. A method of treating and preventing dermatological diseases and conditions, comprising generating and directing a preselected type of light energy from the device of claim 1 to the surface of the skin being treated.

20. The method of claim 19 further comprising maintaining the surface of the skin being treated in a dry condition.

21. The method of claim 19 further comprising applying a photoactivating therapeutic agent to the surface of the skin being treated in combination with directing the light energy thereto.

22. A therapeutic device for treating and preventing dermatological diseases and conditions, the device comprising:
a housing comprising a head portion and a handle portion; and
a moisture reducing assembly located at the head portion, the moisture reducing assembly adapted to generate and direct a controlled air stream flow to the surface of the skin being treated at a temperature and flow rate conducive to safely drying the skin surface.
